(19) United States
(12) Patent Application Publication
(10) Pub. No.: US 2020/0375643 A1
(43) Pub. Date: Dec. 3, 2020

(54) IMPLANT FILLING DEVICE

(71) Applicant: Spineology Inc., St. Paul, MN (US)

(72) Inventors: Todd Bjork, Hudson, WI (US); Steve Jacobson, Lake Elmo, MN (US)

(21) Appl. No.: 16/892,208

(22) Filed: Jun. 3, 2020

Related U.S. Application Data
(60) Provisional application No. 62/856,721, filed on Jun. 3, 2019.

(51) Int. Cl. A61B 17/88

(52) U.S. Cl. CPC A61B 17/8811; A61B 17/8825; A61B 17/8816

(57) ABSTRACT

An implant filling apparatus may include an actuator mechanism, a push rod, straight fill tubes and/or diverted tip fill tubes and an implant connector. An actuator impacts the push rod at a predetermined pressure and rate such that the push rod can drives fill material out of the fill tube(s) and into an implant that is coupled to the implant connector.

### PRIORITY

This application claims the benefit of U.S. Provisional Application Ser. No. 62/856,721, filed on Jun. 3, 2019, which is hereby incorporated herein by reference in its entirety.

### FIELD

The present invention generally relates to surgical instruments. More particularly, the present invention relates to instruments and methods for filling a surgical implant.

### BACKGROUND

It is desirable to access surgical sites through the smallest access possible to minimize disruption to surrounding anatomy. To that end, implants may be inserted through small access openings and then expanded in situ. Expansion may be difficult to control, cumbersome and difficult to reliably reproduce consistently.

### SUMMARY

The present invention is directed toward tools and methods of consistently, easily filling and expanding an implant in situ. In one example, an implant filling apparatus may include an actuator mechanism, a push rod, straight fill tubes and/or diverted tip fill tubes and an implant connector. An actuator impacts the push rod at a predetermined pressure and rate such that the push rod can drives fill material out of the fill tube(s) and into an implant that is coupled to the implant connector.

In another example, an implant filling device can include a fill tube defining an elongated hollow body with a proximal end and an opposing distal end, a push rod sized and shaped to be disposed at least partially within the hollow interior of the fill tube from a proximal end of the fill tube, an implant connector disposed on the distal end of the fill tube, and an actuator configured to engage a proximal end of the push rod.

In another example an implant filling kit can include a surgical implant, a fill material preloaded in a fill tube, wherein the fill tube defining an elongated hollow body with a proximal end and an opposing distal end, a push rod sized and shaped to be disposed at least partially within the hollow interior of the fill tube from a proximal end of the fill tube so that the fill material can be discharged through the distal end of the fill tube, an implant connector disposed on the distal end of the fill tube, and an actuator configured to engage a proximal end of the push rod.

A set of instructions for the surgical method can also be provided in the kit. The kit components can also be contained together in a case or housing. The surgical implant can be a spinal implant or other type of surgical implant that would receive a fill material.

In a further example, a method of filling a surgical implant can include forcibly pushing a push rod through a fill tube with an actuator from a proximal end of the fill tube to discharge a fill material into the surgical implant disposed on a distal end of the fill tube. A force can be supplied from a compressed gas, a wireless electronic drive or from other means to perform the forcible push of the push rod.

The distal end of the fill tube can define a straight tip or a diverted tip. Various types of fill material an be disposed inside of the hollow body of the fill tube. The actuator can be configured to be powered by various means, including, for example, a compressed gas, an electric motor coupled to a battery, and an electric coil. Compressed gas cam be provided by a compressed gas cartridge provided to the actuator.

The detailed technology and preferred embodiments implemented for the subject invention are described in the following paragraphs accompanying the appended drawings for people skilled in this field to well appreciate the features of the claimed invention. It is understood that the features mentioned hereinbefore and those to be commented on hereinafter may be used not only in the specified combinations, but also in other combinations or in isolation, without departing from the scope of the present invention.

### BRIEF DESCRIPTION OF THE DRAWINGS

FIG. 1 is an exploded component diagram for an implant filling instrument in accordance with embodiments of the present invention.

FIG. 2 is a top view of an implant filling instrument in accordance with embodiments of the present invention.

FIG. 3 is a side cross-sectional view of the implant filling instrument of FIG. 2 taken along line A-A in accordance with embodiments of the present invention.

While the invention is amenable to various modifications and alternative forms, specifics thereof have been shown by way of example in the drawings and will be described in detail. It should be understood, however, that the intention is not to limit the invention to the particular example embodiments described. On the contrary, the invention is to cover all modifications, equivalents, and alternatives falling within the spirit and scope of the invention as defined by the appended claims.

### DETAILED DESCRIPTION

In the following descriptions, the present invention will be explained with reference to example embodiments thereof. However, these embodiments are not intended to limit the present invention to any specific example, embodiment, environment, applications or particular implementations described in these embodiments. Therefore, description of these embodiments is only for purpose of illustration rather than to limit the present invention.

It should be appreciated that dimensional relationships among individual elements in the attached drawings are illustrated only for ease of understanding, but not to limit the actual scale.

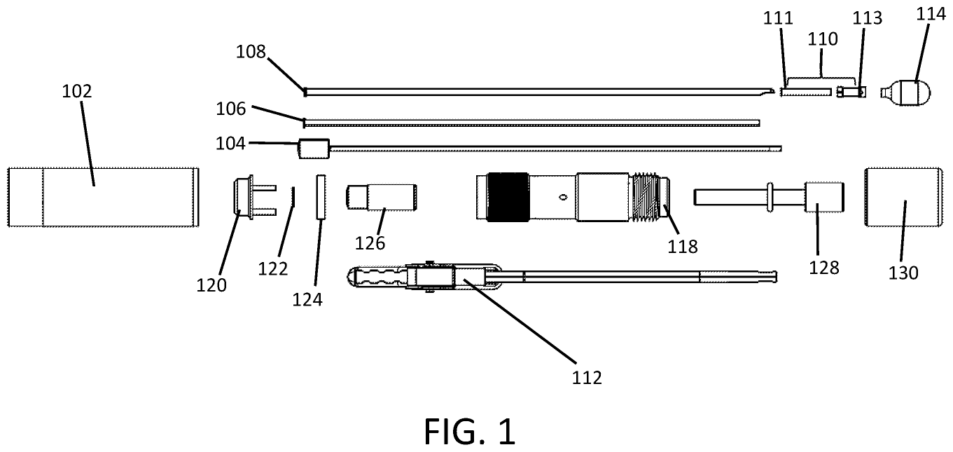

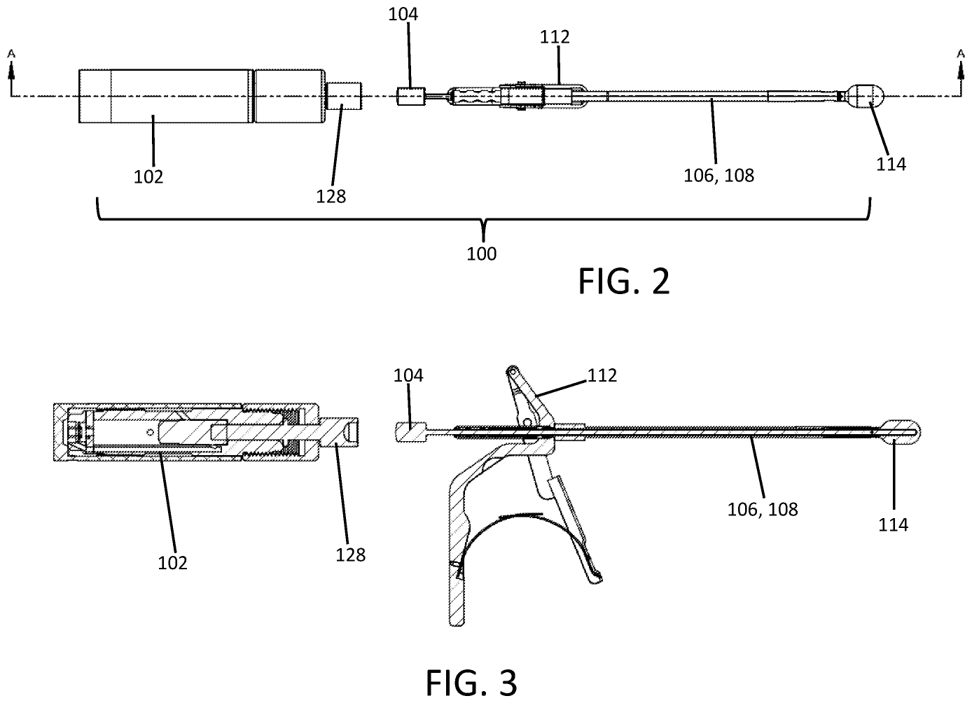

Referring to FIGS. 1-3, an implant filling apparatus 100 according to an example embodiment includes an actuator mechanism 102, a push rod 104, straight fill tubes 106 and/or diverted tip fill tubes 108 and an implant connector 110.

The implant connector 110 in one example can comprise a lock tube 111 and lock tube tip 113.

Fill tubes 106 and 108 may be prefilled with fill material. The fill material used in the present invention may include one or more of the following, or combination thereof, or any other biocompatible material or combination of materials judged to have the desired physiologic response: demineralized bone material, morselized bone graft, cortical, cancellous, or cortico-cancellous, including autograft, allograft, or xenograft; any bone graft substitute or combination of bone graft substitutes, or combinations of bone graft and bone graft substitutes, or bone inducing substances, including but not limited to: calcium phosphates, calcium sulfates, calcium carbonates, hydroxyapatite, bone morphogenic proteins, calcified and/or decalcified bone derivatives; and Bone cements, such as injectable ceramic and polymethylmethacrylate bone cements.

Fill tubes 106 and 108 each have a proximal end, a distal end and an elongated hollow body spanning therebetween. The distal end may define a straight tip 107 (shown on fill tube 106) or a diverted tip 109 (shown on fill tube 108).

Push rod 104 can be inserted into the proximal end of either fill tube 106 or 108. The push rod 104 and fill tube 106 or 108 may then be operably engaged or coupled to the actuator 102.

Filling apparatus 100 may then be positioned through an implant inserter 112 such that the distal end of fill tube 106 or 108 is coupled to the implant 114.

Once the implant 114 is placed into the desired implanting location in the patient, the actuator 102 may be actuated. In an example embodiment, the actuator 102 may be pneumatic, spring loaded, mechanical actuator, AC or DC electric, or any other suitable mechanism to engage and drive push rod 104.

Actuator 102 impacts the push rod 104 at a predetermined pressure and rate such that push rod 104 drives the fill material out of the fill tube 106 or 108 and into the implant 114 at a predictable and consistent flow. In an example embodiment, the filling pressure is optimized such that the pressure is high enough to completely fill implant 114 to a desired fullness, but not too high to overfill implant 114.

The actuator 102 in an example embodiment, comprises an outer housing 116 defining an elongated hollow interior in which an inner housing 118 is disposed. Proximally of the inner housing inside of the outer housing there is disposed an air pop cap 120, air pop diaphragm 122, air pop bushing 124 and shuttle 126. Distally of the inner housing inside of the outer housing there is disposed a plunger 128. A collet 130 is secured to the distal end of the outer housing. The distal end of the plunger 128 is configured to engage the proximal end of the push rod 104. The pop cap 120 contains a pressurized gas or fluid that drives the plunger 128 distally when the pressurized gas or fluid is released.

In another example embodiment, the actuator 102 is cordless and includes a battery powered system that drives a magnetic push rod 104 thru an electrified coil forward and back (distally and proximally) to create an impact, thereby driving fill material out of the fill tube 106 or 108.

According to a further example embodiment, the actuator 102 is cordless and includes a battery powered system that drives an electric motor to actuate the return of the push rod 104 and a switch to activate a release mechanism to allow a spring to drive the push rod 104 forward to create the impact to drive the fill material out of the fill tube 106 or 108.

In another cordless example embodiment, the actuator 102 includes a pressurized gas-driven mechanism that drives push rod 104 forward and back to create the impact that drives the fill material out of the fill tube 106 or 108. The pressurized gas may be provided by a cartridge disposed within or coupled to a hand held driver. The gas may be carbon dioxide, nitrogen or any other compressible inert gas.

In yet another cordless example embodiment, the actuator 102 may include a drive mechanism wherein a battery operates the valves and/or solenoid(s) and a compressible inert gas cartridge is utilized to drive internal push rod 104 forward to create the impact to drive the fill material out of the fill tube 106 or 108.

According to an additional example embodiment, the actuator 102 includes a battery operated hammer drill mechanism providing rotary drilling and hammering impact, which may include two ribbed metal discs engaging against each other, thereby causing an impact force that drives internal push rod 104 forward to create the impact to drive the fill material out of the fill tube 106 or 108.

In another example embodiment, the actuator 102 may include a battery operated rotary hammer mechanism which includes a cylinder of air that is compressed by a piston. The piston then impacts a bit, thereby driving the internal push rod 104 forward to create the impact to drive the fill material out of the fill tube 106 or 108.

The present invention may be embodied in other specific forms without departing from the spirit or essential attributes thereof, and it is, therefore, desired that the present embodiment be considered in all respects as illustrative and not restrictive. Those skilled in the art may recognize other equivalents to the specific embodiment described herein which equivalents are intended to be encompassed by the claims attached hereto.

What is claimed is:

1. An implant filling device, comprising:
a fill tube defining an elongated hollow body with a proximal end and an opposing distal end;
a push rod sized and shaped to be disposed at least partially within the hollow interior of the fill tube from a proximal end of the fill tube;
an implant connector disposed on the distal end of the fill tube; and
an actuator configured to engage a proximal end of the push rod.

2. The implant filling device of claim 1, wherein the distal end of the fill tube defines a straight tip .

3. The implant filling device of claim 1, wherein the distal end of the fill tube defines a diverted tip.

4. The implant filling device of claim 1, wherein fill material is disposed inside of the hollow body of the fill tube.

5. The implant filling device of claim 1, wherein the actuator is configured to be powered by a compressed gas.

6. The implant filling device of claim 1, wherein the actuator is configured to be powered by an electric motor coupled to a battery.

7. The implant filling device of claim 1, wherein the actuator comprises an electric coil to drive the push rod.

8. The implant filling device of claim 1, wherein the actuator comprises a compressed gas cartridge to drive the push rod.

9. An implant filling kit, comprising:
a surgical implant;
a fill material preloaded in a fill tube, the fill tube defining an elongated hollow body with a proximal end and an opposing distal end;
a push rod sized and shaped to be disposed at least partially within the hollow interior of the fill tube from a proximal end of the fill tube so that the fill material can be discharged through the distal end of the fill tube;
an implant connector disposed on the distal end of the fill tube; and
an actuator configured to engage a proximal end of the push rod.

10. The kit of claim 9, wherein the surgical implant is a spinal implant.

11. The implant filling kit of claim 9, wherein the distal end of the fill tube defines a straight tip.

12. The implant filling kit of claim 9, wherein the distal end of the fill tube defines a diverted tip.

13. The implant filling kit of claim 9, wherein fill material is disposed inside of the hollow body of the fill tube.

14. The implant filling kit of claim 9, wherein the actuator is configured to be powered by a compressed gas.

15. The implant filling kit of claim 9, wherein the actuator is configured to be powered by an electric motor coupled to a battery.

16. The implant filling kit of claim 9, wherein the actuator comprises an electric coil to drive the push rod.

17. The implant filling kit of claim 9, herein the actuator comprises a compressed gas cartridge to drive the push rod.

18. A method of filling a surgical implant, the method comprising:
forcibly pushing a push rod through a fill tube with an actuator from a proximal end of the fill tube to discharge a fill material into the surgical implant disposed on a distal end of the fill tube.

19. The method of claim 18, further comprising supplying a force from a compressed gas to perform the forcible push of the push rod.

20. The method of claim 18, further comprising wirelessly supplying an electronic drive force by the actuator to perform the forcible push of the push rod.
